(19) United States
(12) Patent Application Publication
(10) Pub. No.: US 2014/0148492 A1
(43) Pub. Date: May 29, 2014

(54) BICYCLOSUBSTITUTED PYRAZOLE COMPOUNDS FOR TREATING HAIR LOSS

(71) Applicants: Andreas SCHNAPP, Biberach an der Riss (DE); Jeffrey Adam ENCINAS, Ashiya (JP); Andy FOWLER, Guildford (GB); Takeshi KONO, Osaka (JP); Katsuhiro UTO, Higashinada (JP)

(72) Inventors: Andreas SCHNAPP, Biberach an der Riss (DE); Jeffrey Adam ENCINAS, Ashiya (JP); Andy FOWLER, Guildford (GB); Takeshi KONO, Osaka (JP); Katsuhiro UTO, Higashinada (JP)

(73) Assignee: Boehringer Ingelheim International GmbH, Ingelheim am Rhein (DE)

(21) Appl. No.: 14/082,259

(22) Filed: Nov. 18, 2013

(30) Foreign Application Priority Data
Nov. 23, 2012 (EP) 12194118.1

(51) Int. Cl. A61K 8/49; A61Q 7/00

(52) U.S. Cl. CPC A61K 8/494; A61Q 7/00
USPC 514/406

(57) ABSTRACT

The present invention relates to compounds of formula (Ia) or (Ib) or pharmaceutically acceptable salts thereof for use in the treatment or prevention of hairloss,

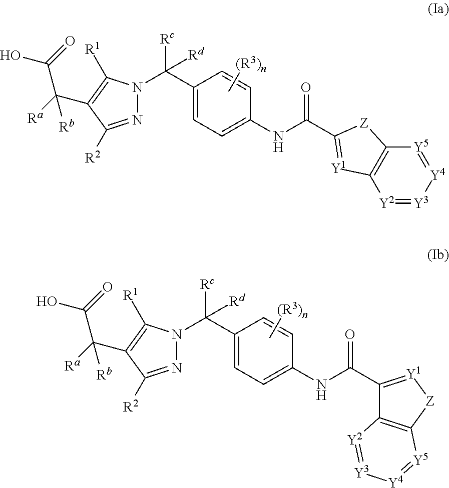

wherein R^{a}, R^{b}, R^{c}, R^{d}, Y¹, Y², Y³, Y⁴, Y⁵, Z, R¹, R², n and R³ have one of the meanings as indicated in the specification and claims, to pharmaceutical compositions containing said compounds or pharmaceutically acceptable salts thereof, to the use of said compounds or pharmaceutically acceptable salts thereof for the manufacture of a medicament useful for the treatment or prevention of hairloss, to a method of treating or preventing hairloss as well as to a method of stimulating hair growth.

The present invention relates to compounds of formula (Ia) or (Ib) or pharmaceutically acceptable salts thereof for use in the treatment or prevention of hairloss,

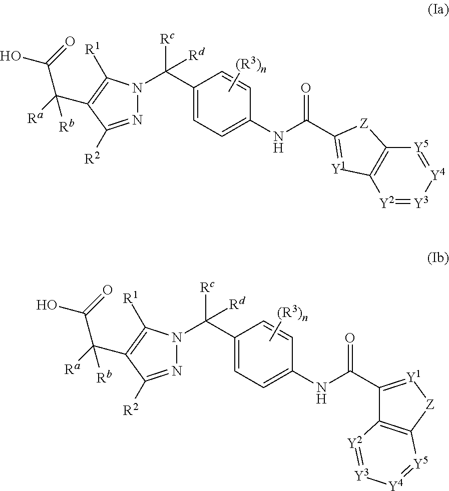

wherein R^{a}, R^{b}, R^{c}, R^{d}, Y², Y³, Y⁴, Y⁵, Z, R¹, R², n and R³ have one of the meanings as indicated in the specification and claims, to pharmaceutical compositions containing said compounds or pharmaceutically acceptable salts thereof, to the use of said compounds or pharmaceutically acceptable salts thereof for the manufacture of a medicament useful for the treatment or prevention of hairloss, to a method of treating or preventing hairloss as well as to a method of stimulating hair growth.

### BACKGROUND OF THE INVENTION

Hair loss has a negative impact on the self-respect of both women and men. The most common type of hairloss effecting both women and men is androgenic alopecia (AGA). In men AGA is characterized by pattern hairloss with bitemporal recession and vertex baldness (MPHL). In women AGA produces female pattern hair loss (FPHL) with a diffuse hairloss over the mid-frontal scalp.

In both forms the hairloss occurs as a result of speckeled hair follicle miniaturization within follicular units and is characterized of hair cycles with a shortened growth (anagen) phase.

In man testosterone is an essential factor involved in the development of AGA. In addition, inflammatory and genetic components have been hypothezised to play a function, too. In women, the impact of androgens on FPHL is unclear. Hair follicle miniaturization and a shortened anagen phase is most likely also the cause of other forms of hair loss.

Treatment options to arrest hair loss progression and stimulate partial hair regrowth include androgen receptor antagonists (spironolactone and cyproterone acetate), the 5α-reductase inhibitor, finasteride, and the androgen-independent hair growth stimulator, minoxidil.

The CRTH2 (chemoattractant receptor-homologous molecule expressed on Th2 cells) protein, also known as GPR44, is a G-protein coupled receptor (GPCR) which is amongst other ligands most strongly activated by prostaglandin D2 (PGD2). PGD2 is a product of prostaglandin D2 synthase (PTGDS).

Garza et al (Science TransMed, 2012, 4, (126):126ra34), showed that PTGDS is upregulated both on the mRNA and protein levels in bald scalp of men with AGA and that PGD2 levels are higher in bald scalp compared to normal scalp, too. Furthermore, they showed that PTGDS protein and PGD2 levels increase before the regression phase of human hair follicles. Administration of PGD2 to explanted human hair follicles inhibited hair growth. When applied topically to the skin of wild-type mice, PGD2 inhibited hair growth, too, but not when applied to CRTH2 (GPR44) knockout mice, suggesting that indeed CRTH2 is the responsible receptor.

Therefore, agents that antagonize the effects of PGD2 at the CRTH2 receptor should be useful for the treatment of AGA, and other forms of hairloss related to enhanced CRTH2 activity.

WO 2007/149312 A2 relates to the use of compounds capable of decreasing the PDG2 level or activity, a downstream signaling or receptor pathway thereof, or PGD2 synthase level or activity, such as CRTH2 antagonists, in methods of treating androgenic alopecia.

It is an objective of the present invention to provide compounds for the treatment of AGA and other conditions of hairloss having enhanced CRTH2 activity or an enhanced PGD2-CRTH2 axis. Said compounds should allow for treating AGA and other forms of hairloss related to enhanced CRTH2 activity.

According to the present invention this objective is achieved by the compounds of formula (Ia) or (Ib) as defined herein and previously disclosed in WO 2012/101043 A1.

### DETAILED DESCRIPTION OF THE INVENTION

The present invention relates to compounds of formula (Ia) or (Ib) or pharmaceutically acceptable salts thereof for use in the treatment or prevention of hairloss, in particular hairloss in humans,

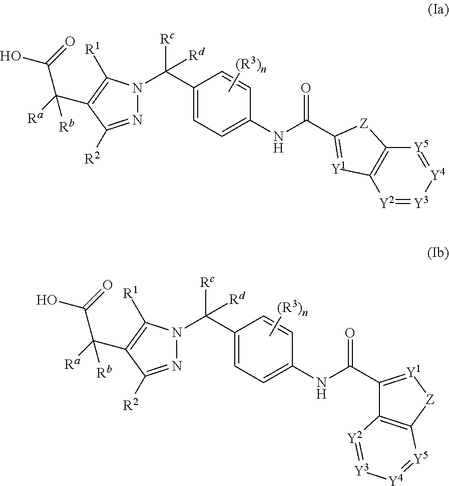

wherein
R^{a} and R^{b} are independently selected hydrogen, hydroxy, halogen, C₁-C₆-alkyl, C₁-C₆ haloalkyl, C₁-C₆-alkoxy, C₁-C₆-haloalkoxy and C₃-C₈-cycloalkyl, or R^{a} and R^{b} together with the carbon atom they are bound to form may form a carbonyl group, or R^{a} and R^{b} together with the carbon atom they are bound to form a 3- to 8-membered ring, wherein said ring may contain 1 or 2 heteroatoms selected from O, N and S as ring member and wherein the ring members of said ring may optionally be independently substituted by hydroxy, halogen, C₁-C₆-alkyl, C₁-C₆-haloalkyl, C₁-C₆-alkoxy, C₁-C₆-haloalkoxy and C₃-C₈-cycloalkyl;
R^{c} and R^{d} are independently selected hydrogen, hydroxy, halogen, C₁-C₆-alkyl, C₁-C₆-haloalkyl, C₁-C₆-alkoxy, C₁-C₆-haloalkoxy and C₃-C₈-cycloalkyl, or R^{c} and R^{d} together with the carbon atom they are bound to form may form a carbonyl group, or R^{c} and R^{d} together with the carbon atom they are bound to form a 3- to 8-membered ring, wherein said ring may contain 1 or 2 heteroatoms selected from O, N and S as ring member and wherein the ring members of said ring may optionally be independently substituted by hydroxy, halogen, C₁-C₆-alkyl, C₁-C₆-haloalkyl, C₁-C₆-alkoxy, C₁-C₆-haloalkoxy and C₃-C₈-cycloalkyl;
Y¹, Y², Y³, Y⁴ and Y⁵ are independently selected from N and CR^{y}, wherein each R^{y} is independently selected from H, hydroxy, halogen, cyano, nitro, SF₅, C(O)NR^{f}R^{g}, C₁-C₆-alkyl, hydroxy-C₁-C₆-alkyl, C₁-C₆-alkoxy-C₁-C₆-alkyl, C₃-C₈-cycloalkyl, C₁-C₆-haloalkyl, C₁-C₆-alkoxy, C₁-C₆-alkoxy-C₁-C₆-alkoxy, C₁-C₆-haloalkoxy, C₃-C₈-cycloalkoxy, C₁-C₆-alkylamino, di-C₁-C₆-alkylamino, C₁-C₆-alkylsulfonyl, phenyl, phenoxy, 5- or 6-membered heterocyclyl and 5- or 6-membered heterocyclyloxy, wherein R^{f} and R^{g} are independently from each other selected from H, C₁-C₆-alkyl, C₁-C₆-haloalkyl, C₃-C₈-cycloalkyl, C₃-C₈-cycloalkenyl and 5- or 6-membered heterocyclyl or R^{f} and R^{g} together with the nitrogen atom to which they are bound form a cyclic amine, which may comprise a further heteroatom selected from O, N and S as a ring member;
Z is selected from O, S and NR^{z}, wherein R^{z} is H, C₁-C₆-alkyl or benzyl;
R¹ and R² are independently from each other selected from H, halogen, C₁-C₆-alkyl, C₂-C₆-alkenyl, C₂-C₆-alkynyl, C₁-C₆-alkoxy, C₁-C₆-alkylthio, C₃-C₈-cycloalkyl, C₃-C₈-cycloalkyl-C₁-C₆-alkyl, C₃-C₈-cycloalkyl-C₂-C₆-alkenyl, C₃-C₈-cycloalkenyl, C₃-C₈-cycloalkenyl-C₁-C₆-alkyl, C₃-C₈-cycloalkenyl-C₂-C₆-alkenyl, phenyl, phenyl-C₁-C₆-alkyl, phenyl-C₂-C₆-alkenyl, naphthyl, naphthyl-C₁-C₆-alkyl, naphthyl-C₂-C₆-alkenyl, heterocyclyl, heterocyclyl-C₁-C₆-alkyl, and heterocyclyl-C₂-C₆-alkenyl, wherein
   the C₁-C₆-alkyl, C₂-C₆-alkenyl and C₂-C₆-alkynyl moieties in the aforementioned radicals R¹ and R² are unsubstituted or carry at least one substituent selected from hydroxy, halogen, cyano, nitro, C₁-C₆-alkoxy, C₁-C₆-haloalkoxy, C₁-C₆-alkylamino, di C₁-C₆-alkylamino and C₁-C6-alkylsulfonyl and/or
   wherein two radicals bound to the same carbon atom of said C₁-C₆-alkyl, C₂-C₆-alkenyl and C₂-C₆-alkynyl moieties in the aforementioned radicals R¹ and R² together with said carbon atom may form a carbonyl group, and wherein
   the C₃-C₈-cycloalkyl, cycloalkenyl, phenyl, naphthyl and heterocyclyl moieties in the aforementioned radicals R¹ and R² are unsubstituted or carry at least one substituent selected from hydroxy, halogen, cyano, nitro, C₁-C₆-alkyl, C₃-C₈-cycloalkyl, C₁-C₆-haloalkyl, C₁-C₆-alkoxy, C₁-C₆-haloalkoxy, C₁-C₆-alkylamino, di-C₁-C₆-alkylamino, C₁-C₆-alkylsulfonyl, phenyl and 5- or 6-membered hetaryl and/or
   wherein two radicals bound to the same carbon atom of said C₃-C₈-cycloalkyl, C₃-C₈-cycloalkenyl and heterocyclyl moieties of the radicals R¹ and R² together with said carbon atom may form a carbonyl group, and wherein
   R^{f} and R^{g} are independently from each other selected from H, C₁-C₆-alkyl, C₁-C₆-haloalkyl, C₃-C₈-cycloalkyl, C₃-C₈-cycloalkenyl and heterocyclyl or
   R^{f} and R^{g} together with the nitrogen atom to which they are bound form a cyclic amine, which may comprise a further heteroatom selected from O, N and S as a ring member;
n is an integer selected from 0, 1, 2 or 3; and
R³ if present are selected independently from each other from halogen, C₁-C₆-alkyl, C₁-C₆-haloalkyl, C₁-C₆-alkoxy, C₁-C₆-haloalkoxy and C₃-C₈-cycloalkyl.

The compounds of formula (Ia) or (Ib) according to the present invention are particularly suitable for treating androgenic alopecia and other forms of hairloss related to enhanced CRTH2 activity.

Accordingly the present invention further relates to pharmaceutical compositions for use in the prevention or treatment of hair loss, in particular hair loss in humans, containing at least one compound of formula (Ia) or (Ib) or a pharmaceutically acceptable salt thereof as defined herein.

The present invention further relates to a method of treating or preventing hair loss, in particular hair loss in a human, said method comprising the step of administering a therapeutically effective amount of at least one compound of formula (Ia) or (Ib) or a pharmaceutically acceptable salt thereof as defined herein.

The present invention further relates to a method of stimulating hair growth, in particular hair growth in a human, said method comprising the step of administering a therapeutically effective amount of at least one compound of formula (Ia) or (Ib) or a pharmaceutically acceptable salt thereof as defined herein.

The present invention further relates to the use of at least one compound of formula (Ia) or (Ib) or a pharmaceutically acceptable salt thereof as defined herein for the manufacture of a medicament useful for the treatment or prevention of hairloss, in particular hairloss in human.

The present invention further relates to the use of at least one compound of formula (Ia) or (Ib) or a pharmaceutically acceptable salt thereof as defined herein for the manufacture of a medicament useful for stimulating hair growth, in particular hair growth in a human.

The activity in a whole cell eosinophil shape change assay of the compounds of the invention can be determined, for example, according to the following references: (i) Mathiesen J M, Ulven T, Martini L, Gerlach L O, Heinemann A, Kostenis E. Identification of indol derivatives exclusively interfering with a G protein-independent signalling pathway of the prostaglandin D2 receptor CRTH2. Mol Pharmacol. 2005 August; 68(2):393-402; (ii) Schuligoi R, Schmidt R, Geisslinger G, Kollroser M, Peskar B A, Heinemann A. PGD2 metabolism in plasma: kinetics and relationship with bioactivity on DP1 and CRTH2 receptors. Biochem Pharmacol. 2007 Jun. 30; 74(1):107-17; (iii) Royer J F, Schratl P, Carrillo J J, Jupp R, Barker J, Weyman-Jones C, Beri R, Sargent C, Schmidt J A, Lang-Loidolt D, Heinemann A. A novel antagonist of prostaglandin D2 blocks the locomotion of eosinophils and basophils. Eur J Clin Invest. 2008 September; 38(9):663-71.

The chemical stability of the compounds of the invention can be determined, for example, under the following conditions: (i) 3 days incubation at 60° C. in 0.1 N HCl (hydrolytic stability under acidc conditions); (ii) 3 days incubation at 60° C. in pH 4.0 buffer solution (hydrolytic stability under weakly acidic conditions); (iii) 3 days incubation at 60° C. in pH 7.4 buffer solution (hydrolytic stability at physiological pH); (iv) 3 days incubation at 20° C. in 0.3% hydrogen peroxide (stability against oxidants); (v) 24 h incubation under UV-radiation (lambda=300-800 nm, P=250 W/m2) in water (stability against light). The kinetics of degradation can, for example, be determined by HPLC analysis.

The pharmacokinetic properties (PK) of the compounds of the invention can be determined in pre-clinical animal species, for example, mouse, rat, dog, guinea pig, mini pig, cynomolgus monkey, rhesus monkey. The pharmacokinetic properties of a compound can be described, for example, by the following parameters: Mean residence time, half-life, volume-of-distribution, AUC (area under the curve), clearance, bioavailability after oral administration.

### USED TERMS AND DEFINITIONS

Terms not specifically defined herein should be given the meanings that would be given to them by one of skill in the art in light of the disclosure and the context. As used in the specification, however, unless specified to the contrary, the following terms have the meaning indicated and the following conventions are adhered to.

In the groups, radicals or moieties defined below, the number of carbon atoms is often specified preceding the group. As an example “C₁-C₆-alkyl” means an alkyl group or radical having 1 to 6 carbon atoms.

In general, for groups comprising two or more subgroups, the last named group is the radical attachment point.

Unless otherwise specified, conventional definitions of terms control and conventional stable atom valences are presumed and achieved in all formulas and groups.

In general all tautomeric forms and isomeric forms and mixtures, whether individual geometric isomers or optical isomers or racemic or non-racemic mixtures of isomers of a chemical structure or compound, are comprised, unless the specific stereochemistry or isomeric form is specifically indicated in the compound name or structure.

The term “substituted” as used herein, means that any one or more hydrogens on the designated atom, moiety or radical is replaced with a selection from the indicated group of radicals, provided that the designated atom's normal valence is not exceeded, and that the substitution results in a stable compound.

The compounds disclosed herein can exist as pharmaceutically acceptable salts. The present invention includes compounds in the form of salts, including acid addition salts. Suitable salts include those formed with both organic and inorganic acids. Such acid addition salts will normally be pharmaceutically acceptable. However, salts of non-pharmaceutically acceptable salts may be of utility in the preparation and purification of the compound in question. Basic addition salts may also be formed and be pharmaceutically acceptable. For a more complete discussion of the preparation and selection of salts, refer to Pharmaceutical Salts: Properties, Selection, and Use (Stahl, P. Heinrich. Wiley-VCH, Zurich, Switzerland, 2002).

The term “pharmaceutically acceptable salt,” as used herein, represents salts or zwitterionic forms of the compounds disclosed herein which are water or oil-soluble or dispersible and pharmaceutically acceptable as defined herein. The salts can be prepared during the final isolation and purification of the compounds or separately by reacting the appropriate compound in the form of the free base with a suitable acid. Representative acid addition salts include acetate, adipate, alginate, L-ascorbate, aspartate, benzoate, benzenesulfonate (besylate), bisulfate, butyrate, camphorate, camphor sulfonate, citrate, digluconate, formate, fumarate, gentisate, glutarate, glycerophosphate, glycolate, hemisulfate, heptanoate, hexanoate, hippurate, hydrochloride, hydrobromide, hydroiodide, 2-hydroxyethansulfonate (isethionate), lactate, maleate, malonate, DL-mandelate, mesitylene sulfonate, methane sulfonate, naphthylene sulfonate, nicotinate, 2-naphthalenesulfonate, oxalate, pamoate, pectinate, persulfate, 3-phenylproprionate, phosphonate, picrate, pivalate, propionate, pyroglutamate, succinate, sulfonate, tartrate, L-tartrate, trichloroacetate, trifluoroacetate, phosphate, glutamate, bicarbonate, para-toluenesulfonate (p-tosylate), and undecanoate. Also, basic groups in the compounds disclosed herein can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. Examples of acids which can be employed to form therapeutically acceptable addition salts include inorganic acids such as hydrochloric acid, hydrobromic acid, sulfuric acid and phosphoric acid, and organic acids such as oxalic acid, maleic acid, succinic acid and citric acid. Salts can also be formed by coordination of the compounds with an alkali metal or alkaline earth ion. Hence, the present invention comprises sodium, potassium, magnesium, and calcium salts of the compounds disclosed herein, and the like.

Basic addition salts can be prepared during the final isolation and purification of the compounds by reacting a carboxy group with a suitable base such as the hydroxide, carbonate, or bicarbonate of a metal cation or with ammonia or an organic primary, secondary, or tertiary amine. The cations of pharmaceutically acceptable salts include lithium, sodium, potassium, calcium, magnesium, and aluminum, as well as nontoxic quaternary amine cations such as ammonium, tetramethylammonium, tetraethylammonium, methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine, and N,N′-dibenzylethylenediamine. Other representative organic amines useful for the formation of base addition salts include ethylenediamine, ethanolamine, diethanolamine, piperidine and piperazine.

While it may be possible for the compounds of the present invention to be administered as the raw chemical, it is also possible to present them as a pharmaceutical formulation. Accordingly, provided herein are pharmaceutical formulations which comprise one or more of certain compounds disclosed herein, or one or more pharmaceutically acceptable salts, esters, prodrugs, amides, or solvates thereof, together with one or more pharmaceutically acceptable carrier and optionally one or more other therapeutic ingredients. The carrier(s) must be “acceptable” in the sense of being compatible with the other ingredients of the formulation and not deleterious to the recipient thereof. Proper formulation is dependent upon the route of administration chosen. Any of the well-known techniques, carriers and excipients may be used as suitable and as understood in the art; e.g. in Remington's Pharmaceutical Sciences. The pharmaceutical compositions disclosed herein may be manufactured in any manner known in the art, e.g., by means of conventional mixing, dissolving, granulating, dragee-making, levigating, emulsifying, encapsulating, entrapping or compression processes.

The term “halogen” as used herein denotes a halogen substituent selected from fluoro, chloro, bromo or iodo.

The term “C₁-C₆-alkyl” as used herein (including the alkyl moieties of C₁-C₆-alkoxy, C₁-C₆-alkylamino, di-C₁-C₆-alkylamino, C₁-C₆-alkylthio and the like) denotes branched and unbranched alkyl moieties with 1 to 6 carbon atoms attached to the remaining compound at any position of the alkyl chain. The term “C₁-C₄-alkyl” accordingly denotes a branched or unbranched alkyl moiety with 1 to 4 carbon atoms. “C₁-C₄-alkyl” is generally preferred. Examples of “C₁-C₆-alkyl” include: methyl, ethyl, n-propyl, iso-propyl, n-butyl, iso-butyl, sec-butyl, tert-butyl, n-pentyl, iso-pentyl, neo-pentyl or hexyl. Unless stated otherwise, the definitions propyl, butyl, pentyl and hexyl include all the possible isomeric forms of the groups in question. Thus, for example, propyl includes n-propyl and iso-propyl, butyl includes iso-butyl, sec-butyl and tert-butyl etc.

The term “C₁-C₆-haloalkyl” as used herein (including the alkyl moieties of C₁-C₆-haloalkoxy, C₁-C₆-haloalkylamino, di-C₁-C₆-haloalkylamino, C₁-C₆-haloalkylthio and the like) denotes branched and unbranched alkyl moieties with 1 to 6 carbon atoms wherein one or more hydrogen atoms are replaced by a halogen atom selected from among fluorine, chlorine or bromine, preferably fluorine and chlorine, particularly preferably fluorine. The term “C₁-C₄-haloalkyl” accordingly denotes branched and unbranched alkyl moieties with 1 to 4 carbon atoms, wherein one or more hydrogen atoms are replaced analogously to what was stated above. C₁-C₄-haloalkyl is generally preferred. Preferred examples include: CH₂F, CHF₂ and CF₃.

The term “C₂-C₆-alkenyl” as used herein (including the alkenyl moieties of other radicals) denotes branched and unbranched alkenyl groups with 2 to 6 carbon atoms attached to the remaining compound at any position of the alkenyl chain and having at least one double bond. The term “C₂-C₄-alkenyl” accordingly denotes branched and unbranched alkenyl moieties with 2 to 4 carbon atoms. Preferred are alkenyl moieties with 2 to 4 carbon atoms.

Examples include: ethenyl or vinyl, propenyl, butenyl, pentenyl or hexenyl. Unless otherwise stated, the definitions propenyl, butenyl, pentenyl and hexenyl include all possible isomeric forms of the moieties in question. Thus, for example, propenyl includes 1-propenyl and 2-propenyl, butenyl includes 1-, 2- and 3-butenyl, 1-methyl-1-propenyl, 1-methyl-2-propenyl etc.

The term “C₂-C₆-alkynyl” as used herein (including the alkynyl moieties of other radicals) denotes branched and unbranched alkynyl groups with 2 to 6 carbon atoms attached to the remaining compound at any position of the alkynyl chain and having at least one triple bond. The term “C₂-C₄-alkynyl” accordingly denotes branched and unbranched alkynyl moieties with 2 to 4 carbon atoms. Alkynyl moieties with 2 to 4 carbon atoms are preferred. Examples include: ethynyl, propynyl, butynyl, pentynyl, or hexynyl. Unless stated otherwise, the definitions propynyl, butynyl, pentynyl and hexynyl include all the possible isomeric forms of the respective moieties. Thus, for example, propynyl includes 1-propynyl and 2-propynyl, butynyl includes 1-, 2- and 3-butynyl, 1-methyl-1-propynyl, 1-methyl-2-propynyl etc.

The term “C₃-C₈-cycloalkyl” as used herein (including the cycloalkyl moieties of other radicals) denotes cyclopropyl, cyclobutyl, cyclopentyl, cyclohexyl, cycloheptyl and cyclooctyl. Preferred are cyclic alkyl groups with 3 to 6 carbon atoms, such as cyclopropyl, cyclopentyl and cyclohexyl.

The term “C₃-C₈-cycloalkenyl” as used herein (including the cycloalkenyl moieties of other radicals) denotes carbocyclic radicals having 3 to 8 carbon atoms and containing at least one, preferably one or two, non-conjugated double bonds. Examples are cyclopentenyl, cyclopantadienyl, cyclohexenyl and cyclohexadienyl.

The term “heterocyclyl” as used herein (including the heterocyclyl moieties of other radicals) denotes 5- to 7-membered heterocyclic radicals and 5- to 10-membered, bicyclic heterocyclic radicals, containing one, two or three heteroatoms, selected from O, N and S as ring members. The heterocyclyl may be linked to the molecule by a carbon atom or, if present, by a nitrogen atom. The term “heterocyclyl” as used herein encompasses saturated or partially unsaturated heterocyclyl as well as hetaryl.

The term “saturated or partially unsaturated heterocyclyl” as used herein (including the heterocyclyl moieties of other radicals) denotes 5- to 7-membered monocyclic heterocyclic radicals as defined above containing a number of double bonds such that no aromatic system is formed as well as 5- to 10-membered bicyclic heterocyclic radicals as defined above containing a number of double bonds such that no aromatic system is formed in at least one of the cycles.

Examples of monocyclic saturated or partially unsaturated heterocyclyl include pyrrolidine, tetrahydrofurane, tetrahydrothiophene, thiazolidine, dioxolane, piperidine, tetrahydropyrane, tetrahydrothiopyrane, piperazine, morpholine, thiomorpholine, oxazepane, and the like.

Examples of bicyclic saturated or partially unsaturated heterocyclyl include dihydropyrrolizine, pyrrolizine, tetrahydroquinoline, tetrahydroisoquinoline, tetrahydroimidazopyridine, tetrahydropyrazolopyridine, benzopyrane, benzodiazepine, and the like.

The term “hetaryl” as used herein (including the heterocyclyl moieties of other radicals) denotes 5- to 7-membered monocyclic heterocyclic radicals as defined above containing a number of double bonds such that an aromatic system is formed as well as 5- to 10-membered bicyclic heterocyclic radicals as defined above containing a number of double bonds such that an aromatic system is formed in both cycles.

Examples of monocyclic aromatic heterocyclyl include furan, thiazole, pyrrole, thiophene, pyrazole, imidazole, thiadiazole, 1,2,3-triazole, 1,2,4-triazole, tetrazole, oxazole, oxadiazole, pyridine, pyridazine, pyrimidine, pyrazine, and the like.

Examples of bicyclic aromatic heterocyclyl include pyrrolizine, indol, indolizine, isoindol, indazol, purine, quinoline, isoquinoline, benzimidazol, benzofuran, benzothiazol, benzoisothiazol, pyridopyrimidine, pteridine, pyrimidopyrimidine, imidazopyridine, pyrazolopyridine, and the like.

The term “fused carbocyclic or heterocyclic moiety” as used herein denotes C₃-C₈-cycloalkyl, C₃-C₈-cycloalkenyl, benzene and heterocyclyl moieties as defined above, wherein said moieties share at least one bond with the cyclic moiety they are bound to. As an example benzene fused to benzene is naphthalene. Preferred are fused cyclic moieties sharing one bond with the cyclic moiety they are fused to. Further preferred the fused moiety is benzene.

The term “3- to 8-membered ring formed by two radicals together with the carbon atom they are bound, wherein said ring may contain 1 or 2 heteroatoms selected from O, N and S as ring member” as used herein denotes C₃-C₈-cycloalkyl, C₃-C₈-cycloalkenyl and heterocyclyl moieties as defined above.

The term “cyclic amine formed by two radicals together with the nitrogen atom to which they are bound, wherein said ring may comprise a further heteroatom selected from O, N and S as a ring member” as used herein denotes cyclic amines having 3 to 8, preferably 5 or 6, ring members. Examples of such formed amines are pyrrolidine, piperidine, piperazine, morpholine, pyrrol, imidazole, and the like.

The terms “heterocyclyl-C₁-C₆-alkyl”, “C₃-C₈-cycloalkyl-C₁-C₆-alkyl”, “phenyl-C₁-C₆-alkyl” and “naphthyl-C₁-C₆-alkyl” as used herein denote alkyl moieties as defined above having 1 to 6 carbon atoms, wherein any one of the hydrogen atoms is replaced by a cyclic moiety as defined above. In these terms the alkyl moiety preferably has 1 to 4 carbon atoms (C₁-C₄-alkyl). More preferably the alkyl moiety is methyl or ethyl, and most preferred methyl. Preferred examples of phenyl-C₁-C₆-alkyl are benzyl or phenethyl.

The terms “heterocyclyl-C₂-C₆-alkenyl”, “C₃-C₈-cycloalkyl-C₂-C₆-alkenyl”, “phenyl-C₂-C₆-alkenyl” and “naphthyl-C₂-C₆-alkenyl” as used herein denote alkenyl moieties as defined above having 2 to 6 carbon atoms, wherein any one of the hydrogen atoms is replaced by a cyclic moiety as defined above. In these terms the alkenyl moiety preferably has 2 to 4 carbon atoms (C₂-C₄-alkenyl). More preferably the alkenyl moiety is ethenyl. A preferred example of phenyl-C₂-C₆-alkenyl is phenethenyl.

The specific and preferred definitions given for the individual radicals and moieties R^{a}, R^{b}, R^{c}, R^{d}, Y¹, Y², Y³, Y⁴, Y⁵, Z, R¹, R², n and R³ herein below are valuable on their own as well as in combination. As will be understood preferred are compounds of formula (Ia) or (Ib) wherein one ore more of the individual radicals and moieties R^{a}, R^{b}, R^{c}, R^{d}, Y¹, Y², Y³, Y⁴, Y⁵, Z, R¹, R², n and R³ have one of the meanings indicated as preferred herein-below and wherein the remaining radicals and moities are as specified hereinbefore. Most preferred are compounds of formula (Ia) or (Ib) wherein all of the individual radicals and moieties R^{a}, R^{b}, R^{c}, R^{d}, Y¹, Y², Y³, Y⁴, Y⁵, Z, R¹, R², n and R³ have one of the meanings indicated as preferred herein-below.

One particular embodiment of the invention relates to pyrazole compounds of formula (Ia), wherein the individual moieties have one of the meanings given in the specification. Preferred are compounds of formula (Ia), wherein the individual moieties have one of the preferred meanings given in the specification.

Another particular embodiment of the invention relates to pyrazole compounds of formula (Ib), wherein the individual moieties have one of the meanings given in the specification. Preferred are compounds of formula (Ib), wherein the individual moieties have one of the preferred meanings given in the specification.

Preferred are pyrazole compounds of formula (Ia) or (Ib), wherein R^{a} and R^{b} are independently selected hydrogen, C₁-C₆-alkyl, C₁-C₆ haloalkyl and C₃-C₈-cycloalkyl.

Particularly preferred are pyrazole compounds of formula (Ia) or (Ib), wherein R^{a} and R^{b} are both hydrogen.

Likewise preferred are pyrazole compounds of formula (Ia) or (Ib), wherein R^{c} and R^{d} are independently selected hydrogen, C₁-C₆-alkyl, C₁-C₆-haloalkyl and C₃-C₈-cycloalkyl.

Particularly preferred are pyrazole compounds of formula (Ia) or (Ib), wherein R^{c} and R^{d} are both hydrogen.

Likewise preferred are pyrazole compounds of formula (Ia) or (Ib), wherein Y¹ is CR^{y1} or N, wherein R^{y1} has one of the meanings given for R^{y}.

More preferred are pyrazole compounds of formula (Ia) or (Ib), wherein Y¹ is CR^{y1}, in particular wherein R^{y1} is selected from H, C₁-C₆-alkyl, C₁-C₆-alkoxy-C₁-C₆-alkyl and C₁-C₆-haloalkyl.

Likewise preferred are pyrazole compounds of formula (Ia) or (Ib), wherein Y² is CR^{y2}, Y³ is CR^{y3}, Y⁴ is CR^{y4} and/or Y⁵ is CR^{y5}, wherein R^{y2}, R^{y3}, R^{y4} and R^{y5} independently from each other have one of the meanings as defined for R^{y}.

More preferred are pyrazole compounds of formula (Ia) or (Ib), wherein Y² is CR^{y2}, Y³ is CR^{y3}, Y⁴ is CR^{y4} and Y⁵ is CR^{y5}, wherein R^{y2}, R^{y3}, R^{y4} and R^{y5} independently from each other have one of the meanings as defined for R^{y}, in particular wherein R^{y2}, R^{y3}, R^{y4} and R^{y5} are independently selected from H, halogen, C₁-C₆-alkoxy, C₁-C₆-alkoxy-C₁-C₆-alkoxy and C₁-C₆-haloalkoxy.

One particular embodiment of the invention relates to pyrazole compounds of formula (Ia) or (Ib), wherein Z is O and the remaining moieties have one of the meanings given in the specification, preferably one of the preferred meanings given in the specification.

Another particular embodiment of the invetion relates to pyrazole compounds of formula (Ia) or (Ib), wherein Z is S and the remaining moieties have one of the meanings given in the specification, preferably one of the preferred meanings given in the specification.

Another particular embodiment of the invention relates to pyrazole compounds of formula (Ia) or (Ib), wherein Z is NR^{z}, wherein RZ is H, C₁-C₆-alkyl or benzyl, and the remaining moieties have one of the meanings given in the specification, preferably one of the preferred meanings given in the specification.

Likewise preferred are pyrazole compounds of formula (Ia) or (Ib), wherein R¹ and R² independently from each other are selected from H, C₁-C₆-alkyl, C₃-C₈-cycloalkyl, phenyl and naphthyl.

More preferred are pyrazole compounds of formula (Ia) or (Ib), wherein R¹ and R² independently from each other are selected from H, C₁-C₄-alkyl, C₃-C₆-cycloalkyl and phenyl.

Particularly preferred are pyrazole compounds of formula (Ia) or (Ib), wherein R¹ and R² are selected from C₁-C₄-alkyl.

Likewise preferred are pyrazole compounds of formula (Ia) or (Ib), wherein n is 0, 1, 2 or 3, in particular wherein n is 0 or 1.

Likewise preferred are pyrazole compounds of formula (Ia) or (Ib), wherein R³ if present are independently selected from halogen, C₁-C₆-alkoxy and C₁-C₆-haloalkoxy.

More preferred are pyrazole compounds of formula (Ia) or (Ib), wherein R³ if present are independently selected from halogen, in particular from F, Cl and Br.

One preferred particular embodiment of the invention relates to pyrazole compounds selected from compounds of formula (Ia′),

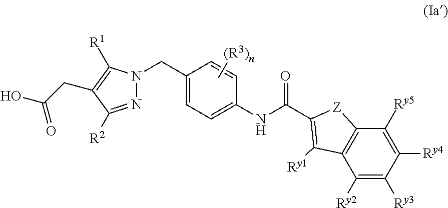

wherein Z, R¹, R², R³, R^{y1}, R^{y2}, R^{y3}, R^{y4} and R^{y5} have one of the meanings given above and n is 0 or 1.

More preferred are pyrazole compounds (Ia′) wherein at least one of the moieties Z, R¹, R², R³, R^{y1}, R^{y2}, R^{y3}, R^{y4} and R^{y5} have one of the preferred meanings given above.

Another preferred particular embodiment of the invention relates to pyrazole compounds selected from compounds of formula (Ib′),

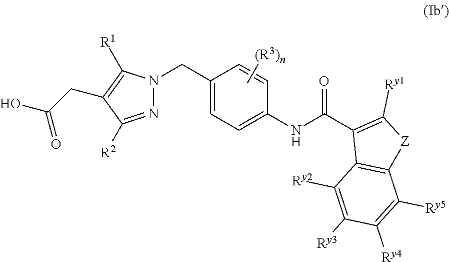

wherein Z, R¹, R², R³, R^{y1}, R^{y2}, R^{y3}, R^{y4} and R^{y5} have one of the meanings given above.

More preferred are pyrazole compounds (Ia′) wherein at leastone of the moieties Z, R¹, R², R³, R^{y1}, R^{y2}, R^{y3}, R^{y4} and R^{y5} have one of the preferred meanings given above.

A further embodiment of the present invention relates to compounds of formula (Ia) or (Ib), wherein the compounds of formula (Ia) or (Ib) are present in the form of the individual optical isomers, mixtures of the individual enantiomers or racemates, preferably in the form of the enantiomerically pure compounds.

A further embodiment of the present invention relates to compounds of formula (Ia) or (Ib), wherein the compounds of formula (Ia) or (Ib) are present in the form of the acid addition salts thereof with pharmacologically acceptable acids as well as optionally in the form of the solvates and/or hydrates.

In a particular embodiment of the invention the hair loss to be treated or prevented is related to androgenic alopecia, in particular to male pattern baldness or to female pattern baldness.

Another aspect of the present invention relates to the compounds of formula (Ia) or (Ib) or pharmaceutically acceptable salts thereof for use for stimulating hair growth, in particular for stimulating hair growth in human.

The at least one compound of formula (Ia) or (Ib) or the pharmaceutically acceptable salt thereof is preferably administered systemically or topically.

### Preparation

The compounds according to the invention may be obtained using methods of synthesis which are known to a person skilled in the art and described in the literature of organic synthesis. Preferably the compounds are obtained in analogy to the methods of preparation explained in more detail in WO 2012/101043 A1.

### Indications/Use

The present invention further relates to the use of at least one compound of formula (Ia) or (Ib) or a pharmaceutically acceptable salt thereof as defined herein for the manufacture of a medicament useful for the treatment or prevention of hairloss.

The at least one compound of formula (Ia) or (Ib) or the pharmaceutically acceptable salt thereof is preferably being used for the treatment or prevention of hairloss related to androgenic alopecia, in particular to male pattern baldness or to female pattern baldness.

According to the present invention the at least one compound of formula (Ia) or (Ib) or the pharmaceutically acceptable salt thereof is preferably used systemically or topically.

Another embodiment of the present invention relates to the manufacturing of a medicament for stimulating hair growth, in particular hair growth in a human. The at least one compound of formula (Ia) or (Ib) or the pharmaceutically acceptable salt thereof is preferably used systemically or topically.

### Method of Treatment

The compounds of formula (Ia) or (Ib) or the pharmaceutically acceptable salt thereof according to the present invention are useful in the prevention and/or treatment of hair loss.

Accordingly the present invention further relates to a method of treating or preventing hairloss, in particular hairloss in a human, said method comprising the step of administering a therapeutically effective amount of at least one compound of formula (Ia) or (Ib) or a pharmaceutically acceptable salt thereof as defined herein.

In a particular embodiment of the present invention the hair loss to be treated or prevented is related to androgenic alopecia, in particular to male pattern baldness or female pattern baldness.

Preferably the at least one compound of formula (Ia) or (Ib) or the pharmaceutically acceptable salt thereof is administered systemically or topically

The compounds of formula (Ia) or (Ib) according to the present invention are also useful for stimulating hair growth.

Accordingly the present invention further relates to a method of stimulating hair growth, in particular hair growth in a human, said method comprising the step of administering a therapeutically effective amount of at least one compound of formula (Ia) or (Ib) or a pharmaceutically acceptable salt thereof as defined herein.

Preferably the at least one compound of formula (Ia) or (Ib) or the pharmaceutically acceptable salt thereof is administered systemically or topically.

### Pharmaceutical Forms

Another aspect of the present invention relates to pharmaceutical compositions for preventing or treating hairloss or for stimulating hairgrowth which are characterized in that they contain a compound of formula (Ia) or (Ib) or a pharmaceutically acceptable salt thereof as defined herein.

Accordingly the present invention relates to a pharmaceutical composition for use in the prevention or treatment of hairloss, in particular hair loss in humans, containing at least one compound of formula (Ia) or (Ib) or a pharmaceutically acceptable salt thereof as defined herein.

Another embodiment of the present invention relates to pharmaceutical composition for use in method of stimulating hair growth, in particular hair growth in a human, said method comprising the step of administering a therapeutically effective amount of at least one compound of formula (Ia) or (Ib) or a pharmaceutically acceptable salt thereof as defined herein.

The pharmaceutical compositions of the present invention may preferably be administerd systemically or topically. Accordingly the present invention further relates to suitable topical or systemical pharmaceutical formulations for use in the novel methods of treatment and uses of the present invention.

The dosage of the compounds according to the invention is naturally highly dependent on the method of administration.

The term “therapeutically effective amount” as used herein refers to a daily dosage of the compounds of formula (Ia) or (Ib) or the pharmaceutically acceptable salts thereof in a range from 0.5 to 1000 mg, preferably 1 to 500 mg, more preferably from 5 to 200 mg.

The content of the pharmaceutically active compound(s) should be in the range from 0.05 to 90 wt.-%, preferably 0.1 to 50 wt.-% of the total weight of the pharmaceutical composition.

The pharmaceutical compositions containing the compound or composition may be administered to a subject by any method known to a person skilled in the art, e.g. parenterally, transdermally, intravenously, intradermally or subcutaneously.

The pharmaceutical compositions containing the compounds of the present invention can be administered in a wide variety of therapeutic dosage forms in conventional vehicles for systemic administration, as for example, by oral administration in the form of tablets, capsules, solutions, suspensions, suppositories, powders or by intravenous injection.

One particular embodiment of the present invention relates to pharmaceutical composition as defined herein, wherein the pharmaceutical composition is a tablet, capsule, pill, solution, suspension, dispersion, emulsion or suppository for systemical administration.

In another embodiment, the pharmaceutical composition is administered topically to the body surfaces and is thus formulated in a form suitable for topical administration.

Suitable topical formulations include gels, ointments, creams, lotions, drops and the like.

Another particular embodiment of the present invention therefor relates to the pharmaceutical composition as defined herein, wherein the pharmaceutical composition is a solution, suspension, emulsion, dispersion, cream, ointment, gel, lotion, shampoo or aerosol for topical administration.

Solutions are prepared in the usual way, e.g. with the addition of isotonic agents, preservatives such as p-hydroxybenzoates or stabilizers such as alkali metal salts of ethylenediaminetetraacetic acid, optionally using emulsifiers and/or dispersants, while if water is used as diluent, for example, organic solvents may optionally be used as solubilisers or dissolving aids, and the solutions may be transferred into injection vials or ampoules or infusion bottles.

Suitable tablets may be obtained, for example, by mixing the active substance(s) with known excipients, for example inert diluents such as calcium carbonate, calcium phosphate or lactose, disintegrants such as corn starch or alginic acid, binders such as starch or gelatine, lubricants such as magnesium stearate or talc and/or agents for delaying release, such as carboxymethyl cellulose, cellulose acetate phthalate, or polyvinyl acetate. The tablets may also comprise several layers.

Coated tablets may be prepared accordingly by coating cores produced analogously to the tablets with substances normally used for tablet coatings, for example collidone or shellac, gum arabic, talc, titanium dioxide or sugar. To achieve delayed release or prevent incompatibilities the core may also consist of a number of layers. Similarly the tablet coating may consist of a number or layers to achieve delayed release, possibly using the excipients mentioned above for the tablets.

Syrups or elixirs containing the active substances or combinations thereof according to the invention may additionally contain a sweetener such as saccharine, cyclamate, glycerol or sugar and a flavor enhancer, e.g. a flavoring such as vanillin or orange extract. They may also contain suspension adjuvants or thickeners such as sodium carboxymethyl cellulose, wetting agents such as, for example, condensation products of fatty alcohols with ethylene oxide, or preservatives such as p-hydroxybenzoates.

Capsules containing one or more active substances or combinations of active substances may for example be prepared by mixing the active substances with inert carriers such as lactose or sorbitol and packing them into gelatine capsules.

Suitable suppositories may be made for example by mixing with carriers provided for this purpose, such as neutral fats or polyethyleneglycol or the derivatives thereof.

Excipients which may be used include but are not limited to water, pharmaceutically acceptable organic solvents such as paraffins (e.g. petroleum fractions), vegetable oils (e.g. groundnut or sesame oil), mono- or polyfunctional alcohols (e.g. ethanol or glycerol), carriers such as e.g. natural mineral powders (e.g. kaolins, clays, talc, chalk), synthetic mineral powders (e.g. highly dispersed silicic acid and silicates), sugars (e.g. cane sugar, lactose and glucose), emulsifiers (e.g. lignin, spent sulphite liquors, methylcellulose, starch and polyvinylpyrrolidone) and lubricants (e.g. magnesium stearate, talc, stearic acid and sodium lauryl sulphate).

For oral use the tablets may obviously contain, in addition to the carriers specified, additives such as sodium citrate, calcium carbonate and dicalcium phosphate together with various additional substances such as starch, preferably potato starch, gelatine and the like. Lubricants such as magnesium stearate, sodium laurylsulphate and talc may also be used to produce the tablets. In the case of aqueous suspensions the active substances may be combined with various flavor enhancers or colorings in addition to the abovementioned excipients.

The preferred excipients include antioxidants such as ascorbic acid, for example, provided that it has not already been used to adjust the pH, vitamin A, vitamin E, tocopherols and similar vitamins and provitamins occurring in the human body.

Preservatives may be used to protect the formulation from contamination with pathogens. Suitable preservatives are those which are known in the art, particularly cetyl pyridinium chloride, benzalkonium chloride or benzoic acid or benzoates such as sodium benzoate in the concentration known from the prior art. The preservatives mentioned above are preferably present in concentrations of up to 50 mg/100 ml, more preferably between 5 and 20 mg/100 ml.

The pharmaceutical compositions provided herein may be formulated as controlled-release compositions or as immediate-release composition.

The following examples serve to further illustrate the present invention without restricting its scope.

### Examples

### I. HPLC Methods

### Method A:

### HPLC-MS: Agilent 1100

### Mobile Phase:

A: water with 0.032% NH₄OH
B: methanol

| time in min | % A | % B | flow rate in ml/min |
|---|---|---|---|
| 0.00 | 95 | 5 | 1.50 |
| 2.00 | 0 | 100 | 1.50 |
| 2.50 | 0 | 100 | 1.50 |
| 2.60 | 95 | 5 | 1.50 |
| 2.90 | 95 | 5 | 1.50 |

Column: XBridge C18, 3.5 μm, 4.6×50 mm (column temperature: constant at 40° C.).
Detection by diode array detector at 210-500 nm wavelength.

### Method B:

HPLC-MS: Waters ZQ MS, Alliance 2690/2695 HPLC, 2996 diode array detector

### Mobile Phase:

A: water with 0.1% TFA
B: methanol

| time in min | % A | % B | flow rate in ml/min |
|---|---|---|---|
| 0.00 | 95 | 5 | 4.0 |
| 0.20 | 95 | 5 | 4.0 |
| 1.60 | 0 | 100 | 4.0 |
| 2.10 | 0 | 100 | 4.0 |

Column: Waters XBridge C18, 4.6×20 mm, 3.5 μm (column temperature: constant at 40° C.).
Detection by diode array detector at 210-400 nm wavelength.

### Method C:

HPLC: Waters Acquity with DA and MS detector

### Mobile Phase:

A: water with 0.1% TFA
B: methanol

| time in min | % A | % B | flow rate in ml/min |
|---|---|---|---|
| 0.00 | 99 | 1 | 1.5 |
| 0.05 | 99 | 1 | 1.5 |
| 1.05 | 0 | 100 | 1.5 |
| 1.20 | 0 | 100 | 1.5 |

Column: Waters XBridge BEH C18, 2.1×30 mm, 1.7 μm (column temperature: constant at 60° C.). Detection by diode array detector at 210-400 nm wavelength.

### Method D:

HPLC: Waters Acquity with DA and MS detector

### Mobile Phase:

A: water with 0.13% TFA
B: methanol with 0.05% TFA

| time in min | % A | % B | flow rate in ml/min |
|---|---|---|---|
| 0.00 | 99 | 1 | 1.3 |
| 0.05 | 99 | 1 | 1.3 |
| 1.05 | 0 | 100 | 1.3 |
| 1.20 | 0 | 100 | 1.3 |

Column: Waters XBridge BEH C18, 2.1×30 mm, 1.7 μm (column temperature: constant at
60° C.). Detection by diode array detector at 210-400 nm wavelength.

### Method E:

HPLC: Waters Acquity with DA and MS detector

### Mobile Phase:

A: water with 0.1% TFA
B: methanol

| time in min | % A | % B | flow rate in ml/min |
|---|---|---|---|
| 0.00 | 95 | 5 | 1.4 |
| 0.05 | 95 | 5 | 1.4 |
| 1.00 | 0 | 100 | 1.4 |
| 1.10 | 0 | 100 | 1.4 |

Column: Waters XBridge C18, 2.1×30 mm, 2.5 μm (column temperature: constant at 60° C.).
Detection by diode array detector at 210-400 nm wavelength.

### Method F:

HPLC: Agilent 1200 with DA and MS detector

### Mobile Phase:

A: water with 0.1% TFA
B: methanol

| time in min | % A | % B | flow rate in ml/min |
|---|---|---|---|
| 0.00 | 95 | 5 | 2.0 |
| 0.20 | 95 | 5 | 2.0 |
| 1.50 | 0 | 100 | 2.0 |
| 1.55 | 0 | 100 | 2.6 |
| 1.75 | 0 | 100 | 2.6 |

Column: Waters XBridge C18, 3×30 mm, 2.5 μm (column temperature: constant at 60° C.).
Detection by diode array detector at 210-400 nm wavelength.

### Method G:

HPLC-MS: Waters Alliance with DA and MS detector

### Mobile Phase:

A: water with 0.1% NH₃
B: methanol with 0.1% NH₃

| time in min | % A | % B | flow rate in ml/min |
|---|---|---|---|
| 0.00 | 95 | 5 | 4.0 |
| 0.20 | 95 | 5 | 4.0 |
| 1.50 | 0 | 100 | 4.0 |
| 1.75 | 0 | 100 | 4.0 |

Column: Waters XBridge C18, 4.6×30 mm, 3.5 μm (column temperature: constant at 60° C.).
Detection by diode array detector at 210-400 nm wavelength.

### Method H:

HPLC-MS: Waters Alliance with DA and MS detector

### Mobile Phase:

A: water with 0.1% TFA
B: methanol

| time in min | % A | % B | flow rate in ml/min |
|---|---|---|---|
| 0.00 | 95 | 5 | 4.8 |
| 1.60 | 0 | 100 | 4.8 |
| 1.85 | 0 | 100 | 4.8 |
| 1.90 | 95 | 5 | 4.8 |

Column: Waters SunFire C18, 4.6×30 mm, 3.5 μm (column temperature: constant at 60° C.).
Detection by diode array detector at 210-400 nm wavelength.

### Method J:

HPLC: Waters Acquity with DA and MS detector

### Mobile Phase:

A: water with 0.13% TFA
B: methanol with 0.05% TFA

| time in min | % A | % B | flow rate in ml/min |
|---|---|---|---|
| 0.00 | 99 | 1 | 1.2 |
| 0.15 | 99 | 1 | 1.2 |
| 1.10 | 0 | 100 | 1.2 |
| 1.25 | 0 | 100 | 1.2 |

Column: Waters Sunfire C18, 2.1×30 mm, 2.5 μm (column temperature: constant at 60° C.).
Detection by diode array detector at 210-400 nm wavelength.

### Method K:

HPLC-MS: Waters Alliance with DA and MS detector

### Mobile Phase:

A: water with 0.1% TFA
B: methanol with 0.1% TFA

| time in min | % A | % B | flow rate in ml/min |
|---|---|---|---|
| 0.00 | 95 | 5 | 4.0 |
| 0.20 | 95 | 5 | 4.0 |
| 1.50 | 0 | 100 | 4.0 |
| 1.75 | 0 | 100 | 4.0 |
| 1.85 | 95 | 5 | 4.0 |

Column: Waters XBridge C18, 4.6×30 mm, 3.5 μm (column temperature: constant at 60° C.).
Detection by diode array detector at 210-400 nm wavelength.

### Method L:

HPLC-MS: Waters Alliance with DA and MS detector

### Mobile Phase:

A: water with 0.1% TFA
B: methanol

| time in min | % A | % B | flow rate in ml/min |
|---|---|---|---|
| 0.00 | 95 | 5 | 4.8 |
| 1.60 | 0 | 100 | 4.8 |
| 1.85 | 0 | 100 | 4.8 |
| 1.90 | 95 | 5 | 4.8 |

Column: Waters XBridge C18, 4.6×30 mm, 3.5 μm (column temperature: constant at 60° C.).
Detection by diode array detector at 210-400 nm wavelength.

### Method M:

HPLC-MS: Waters 2695 HPLC, ZQ MS, 2996 diode array detector, 2695 autosampler

### Mobile Phase:

A: water with 0.1% NH₃
B: methanol with 0.1% NH₃

| time in min | % A | % B | flow rate in ml/min |
|---|---|---|---|
| 0.00 | 95 | 5 | 4.0 |
| 0.20 | 95 | 5 | 4.0 |
| 1.50 | 0 | 100 | 4.0 |
| 1.75 | 0 | 100 | 4.0 |

Column: Waters XBridge C18, 4.6×30 mm, 3.5 μm (column temperature: constant at 60° C.).
Detection by diode array detector at 210-400 nm wavelength.

### Method N:

HPLC: Waters Acquity with DA and MS detector

### Mobile Phase:

A: water with 0.13% TFA
B: methanol with 0.08% TFA

| time in min | % A | % B | flow rate in ml/min |
|---|---|---|---|
| 0.00 | 99 | 1 | 1.3 |
| 0.05 | 99 | 1 | 1.3 |
| 0.35 | 0 | 100 | 1.3 |
| 0.50 | 0 | 100 | 1.3 |

Column: Waters XBridge BEH C18, 2.1×30 mm, 1.7 μm (column temperature: constant at
60° C.). Detection by diode array detector at 210-400 nm wavelength.

### Method O:

HPLC: Agilent 1200 with DA and MS detector

### Mobile Phase:

A: water with 0.1% TFA
B: methanol

| time in min | % A | % B | flow rate in ml/min |
|---|---|---|---|
| 0.00 | 95 | 5 | 1.9 |
| 0.20 | 95 | 5 | 1.9 |
| 1.55 | 0 | 100 | 1.9 |
| 1.60 | 0 | 100 | 2.4 |
| 1.80 | 0 | 100 | 2.4 |

Column: Waters XBridge C18, 3×30 mm, 2.5 μm (column temperature: constant at 60° C.).
Detection by diode array detector at 210-400 nm wavelength.

### IV) Biological Assays

The compounds of formula (Ia) and (Ib) according to the invention were tested using the following biological test methods to determine their ability to displace PGD₂ from the CRTH2 receptor and for their ability to antagonise the functional effects of PGD₂ at the CRTH2 receptor in a whole system.

### Preparation of Human CRTH2 Receptor Membranes and Radioligand Binding Assay

The binding of CRTH2 antagonists is determined using membranes prepared from Chinese hamster ovary cells (CHO-K1 cells) transfected with the human CRTH2 receptor (CHO-K1-hCRTH2 cells, Perkin Elmer, Cat No ES-561-C). To produce cell membranes the CHO-K1-hCRTH2 cells are cultured in suspension in CHO SFMII medium supplemented with 400 μg/ml G418. The cells are harvested by centrifugation at 300 g for 10 min at room temperature. The cell pellet is resuspended in Phosphate Buffer Saline (PBS) including a protease inhibitor mix (Complete, Roche) and adjusted to a concentration of 10E7 cells/ml. The CHO-K1-hCRTH2 cells are disrupted by nitrogen decomposition to obtain the membrane preparation. Cell debris is removed by centrifugation (500 g at 4° C., 30 min) and the supernatant is transferred into fresh tubes followed by a second centrifugation at 40000 g for 1 h at 4° C. to sediment the membranes. The membranes are suspended in SPA incubation buffer (50 mM Tris HCl, 10 mM MgCl₂, 150 mM NaCl, 1 mM EDTA, pH 7.4) without bovine serum albumin, homogenized by passing through a single use needle (Terumo, 23G×1″), and stored in aliquots at −80° C.

The CRTH2 receptor binding assay is performed in a scintillation proximity assay (SPA) format with the radioligand [³H]-PGD₂ (Perkin Elmer, NET616000MC). CHO-K1-hCRTH2 cell membranes are again homogenized by passing through a single use needle (Terumo, 23G×1″) and diluted in SPA incubation buffer in suitable concentrations (0.5-10 μg protein/well). The SPA assay is set up in 96 well microtiter plates (Perkin Elmer, CatNo. 6005040) in SPA incubation buffer with a final volume of 200 μl per well and final concentration of 50 mM Tris-HCl, 10 mM MgCl₂, 150 mM NaCl, 1 mM EDTA pH 7.4, 0.1% bovine serum albumin). The SPA assay mixture contains 60 μl of the membrane suspension, 80 μl of Wheat Germ Agglutinin coated PVT beads (GE Healthcare, RPNQ-0001, 0.3 mg/well), 40 μl of [3H]-PGD₂ diluted in SPA buffer to a final concentration of 1 nM (50 000 dpm) and 20 μl of the test compound (dissolved in dimethylsulfoxid). The SPA assay mixture is incubated for 3 h at room temperature. Bound radioactivity is determined with a scintillation counter (Micro Beta Trilux, Wallac).

The binding of [³H]-PGD₂ to CHO-K1-hCRTH2 cell membranes is determined in the absence (total binding, Bo) and presence (non-specific binding, NSB) of unlabelled PGD₂ (1 μM, Cayman Chemical, Cat No 12010) or a reference CRTH2 antagonist (10 μM CAY10471, Cayman Chemical, Cat No 10006735).

Determination of the affinity of a test compound is calculated by subtraction of the non-specific binding (NSB) from the total binding (Bo) or the binding in the presence of the test compound (B) at a given compound concentration. The NSB value is set to 100% inhibition. The Bo-NSB value is set to 0% inhibition.

% inhibition values were obtained at a defined compound concentration, e.g. at 1 μM, % inhibition of the test compound was calculated by the formula 100−((B−NSB)*100/(Bo−NSB)). % inhibition values above 100% are founded by assay variance.

The dissociation constant Kᵢ was calculated by iterative fitting of experimental data obtained at several compound concentrations over a dose range from 0.1 to 30 000 nM using the law of mass action based program “easy sys” (Schittkowski, Num Math 68, 129-142 (1994)).

### CRTH2 Camp Functional Assay Protocol

The assay is conducted in CHO-K1-hCRTH2 cells. Intracellular cAMP is generated by stimulating the cells with 10 μM Forskolin, an adenylate cyclase activator. PGD2 is added to activate the CRTH2 receptor which results in the attenuation of the forskolin-induced cAMP generation. Test compounds are tested for their ability to inhibit the PGD2-mediated attenuation of the Forskolin-induced cAMP generation in CHO-K1-hCRTH2 cells. CHO-K1-hCRTH2 cells are cultured in roller bottles in CHO SFMII medium supplemented with 400 μg/ml G418. The cells are harvested by centrifugation at 300 g for 10 min at room temperature. The cell pellet is washed and suspended in PBS. The cells are adjusted to a final concentration of 4×10E6 cells/ml.

Test compounds are diluted in dimethylsulfoxid and tested at several compound concentrations over a dose range from 0.1 to 3 000 nM.

The cAMP levels are determined by an AlphaScreen cAMP assay (Perkin Elmer CatNo. 6760625M) in 384 well optiplates (PerkinElmer, CatNo. 6007290) with a total assay volume of 50 μl. 10 μl of cells (40.000 cells per well) are incubated for 30 min at 37° C. with 10 μl of a stimulation mix containing a final concentration of 10 μM Forskolin, 30 nM PGD2, 0.5 mM IBMX, 5 mM HEPES, 1×HBSS buffer, 0.1% BSA, adjusted to pH 7.4, and the test compound at various concentrations. Thereafter, 30 μl of a lysis and detection mix is added containing SA donor beads, biotinylated cAMP, anti-cAMP acceptor beads, 0.3% Tween-20, 5 mM HEPES, 0.1% BSA, adjusted to pH 7.4. After 2 h incubation time the AlphaScreen signal is read on an AlphaQuest-HTS instrument. The IC₅₀ values are calculated by using the Prism software.

### Other CRTH2 Functional Assay Protocols

The ability of the tested compounds to antagonise the functional effects of PGD2 at the CRTH2 receptor may also be demonstrated by methodology known in the art, such as by a whole cell binding assay, a GTPgS assay, a BRET assay, an inositol phosphate accumulation assay, an CRTH2 cell surface expression assay, a Ca²⁺ influx assay, an ERK phosphorylation assay, an cell migration assay, an eosinophil shape change assay, a Th2 cell degranulation assay, or a basophil activation assay as described by Mathiesen et al., Mol Pharmacol. 2005, 68:393-402; Mimura et al., J. Pharmacol. Exp. Ther., 2005, 314:244-51; Sandham et al., Bioorg. Med. Chem. Lett., 2007, 17:4347-50; Sandham Bioorg. Med. Chem. Lett., 2009, 19:4794-8; Crosignani et al., J Med Chem, 2008, 51:2227-43; Royer et al., Eur J Clin Invest, 2008, 38:663-71; Boehme et al., Int Immunol, 2009, 21:621-32; Sugimoto et al., Pharmacol. Exp. Ther., 2003, 305:347-52; Monneret et al., J Pharmacol Exp Ther, 2005, 312:627-34; Xue et al., J. Immunol., 2005, 175:6531-6.

Cell lines for expressing the CRTH2 receptor include those naturally expressing the CRTH2 receptor, such as AML14.3D10 and NCI-H292 cells (Sawyer et al., Br. J. Pharmacol., 2002, 137:1163-72; Chiba et al., Int. Arch. Allergy. Immunol., 2007, 143 Suppl 1:23-7), those induced to express the CRTH2 receptor by the addition of chemicals, such as HL-60 or AML14.3D10 cells treated with, for example, butyric acid (Sawyer et al., Br. J. Pharmacol., 2002, 137:1163-72) or a cell line engineered to express a recombinant CRTH2 receptor, such as L1.2, CHO, HEK-293, K562 or CEM cells (Liu et al., Bioorg. Med. Chem. Lett., 2009, 19:6840-4; Sugimoto et al., Pharmacol Exp Ther, 2003, 305:347-52; Hata et al., Mol. Pharmacol., 2005, 67:640-7; Nagata et al., FEBS Lett, 1999, 459:195-9).

Finally, blood or tissue cells, for example human peripheral blood eosinophils, isolated using methods as described by Hansel et al., J. Immunol. Methods., 1991, 145, 105-110, or human Th2 cells isolated and treated as described by Xue et al., J. Immunol., 2005, 175:6531-6, or human basophils isolated and characterized as described by Monneret et al., J. Pharmacol. Exp. Ther., 2005, 312:627-34 can be utilized in such assays.

In particular, the compounds of the present invention have activity in binding to the CRTH2 receptor in the aforementioned assays and inhibit the activation of CRTH2 by CRTH2 ligands. As used herein, “activity” is intended to mean a compound demonstrating an inhibition of 50% at 1 μM or higher in inhibition, or a Kᵢ value<1 μM, when measured in the aforementioned assays. Such a result is indicative of the intrinsic activity of the compounds as inhibitor of CRTH2 receptor activity. Antagonistic activities of selected compounds are shown in tables 1 and 2 below.

TABLE 1
Compounds of formula Ia″

(Ia″)

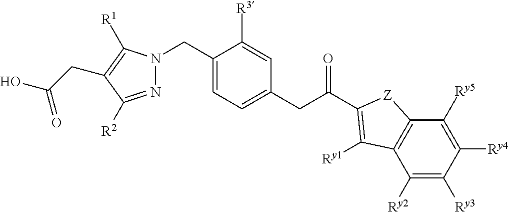

| | | | | R^{y1}; R^{y2}; R^{y3}; | MS | Retention | Ki |
|---|---|---|---|---|---|---|---|
| Cmpd | R^{3′} | R¹ = R² | Z | R^{y4}; R^{y5} | [M + H]⁺ | Time | [nM] |
| 1 | F | CH₃ | NH | H; H; H; | 421 | 1.04 | 0.8 |
| | | | | H; H | | method G | |
| 2 | Cl | CH₃ | NH | H; H; H; | 437 | 1.36 min | 0.5 |
| | | | | H; H | | method B | |
| 3 | F | CH₃ | NCH₂— | H; H; H; | 511 | 0.98 min | 0.2 |
| | | | C₆H₅ | H; H | | method D | |
| 4 | H | CH₃ | O | CH₃; H; F; | 436 | 0.85 min | 0.8 |
| | | | | H; H | | method D | |
| 5 | Cl | CH₃ | O | H; H; H; | 438 | 0.86 min | 0.8 |
| | | | | H; H | | method D | |
| 6 | H | CH₃ | O | H; H; OCH₃; | 434 | 0.77 min | 0.6 |
| | | | | H; H | | method D | |
| 7 | H | CH₃ | O | CH₃; H; H; | 436 | 0.86 min | 1.3 |
| | | | | H; F | | method D | |
| 8 | H | CH₃ | O | H; H; Cl; | 438 | 0.84 min | 0.3 |
| | | | | H; H | | method D | |
| 9 | H | CH₃ | O | H; H; H; | 434 | 0.78 min | 1.2 |
| | | | | H; OCH₃ | | method D | |
| 10 | H | CH₃ | NH | H; H; H; | 403 | 1.24 min | 0.2 |
| | | | | H; H | | method B | |
| 11 | H | CH₃ | NH | H; H; H; | 421 | 0.76 min | 0.2 |
| | | | | F; H | | method D | |
| 12 | H | CH₃ | NH | H; H; F; | 421 | 0.76 min | 0.2 |
| | | | | H; H | | method D | |
| 13 | H | CH₃ | NCH₃ | CH₃; H; H; | 431 | 0.81 min | 63.9 |
| | | | | H; H | | method D | |
| 14 | H | CH₃ | NH | CH₃; H; OCH₃; | 447 | 0.79 min | 0.1 |
| | | | | H; H | | method D | |
| 15 | H | CH₃ | S | H; H; H; H; H | 420 | 1.32 min | 0.3 |
| | | | | | | method B | |
| 16 | H | CH₃ | NCH₃ | H; H; H; | 417 | 1.29 min | 0.8 |
| | | | | H; H | | method K | |
| 17 | H | CH₃ | NC₂H₅ | H; H; H; | 431 | 1.35 min | 0.4 |
| | | | | H; H | | method K | |
| 18 | H | CH₃ | NCH₂— | H; H; H; | 493 | 0.88 min | <0.1 |
| | | | C₆H₅ | H; H | | method D | |
| 19 | H | C₂H₅ | O | H; H; Cl; | 466 | 1.43 min | 0.2 |
| | | | | H; H | | method K | |
| 20 | H | C₂H₅ | NH | H; H; H; | 431 | 1.30 min | 0.3 |
| | | | | H; H | | method K | |
| 21 | H | C₂H₅ | NCH₃ | H; H; H; | 445 | 0.84 min | 0.3 |
| | | | | H; H | | method D | |
| 22 | H | C₂H₅ | NC₂H₅ | H; H; H; | 459 | 0.87 min | 0.2 |
| | | | | H; H | | method D | |
| 23 | H | C₂H₅ | NH | H; H; F; | 449 | 0.80 min | 0.3 |
| | | | | H; H | | method D | |
| 24 | H | C₂H₅ | NCH₃ | CH₃; H; H; | 459 | 0.84 min | 9.9 |
| | | | | H; H | | method D | |
| 25 | H | C₂H₅ | NH | H; H; H; | 449 | 1.34 min | 0.2 |
| | | | | F; H | | method K | |
| 26 | H | CH₃ | O | H; H; H; | 404 | 1.28 min | 0.8 |
| | | | | H; H | | method B | |
| 27 | F | CH₃ | O | CH₃; H; F; | 454 | 0.90 min | 0.6 |
| | | | | H; H | | method D | |
| 28 | H | CH₃ | N(CH₂)₂— | H; H; H; | 463 | 0.85 min | 0.1 |
| | | | CH₃ | F; H | | method E | |
| 29 | H | CH₃ | NC₂H₅ | H; H; H; | 449 | 0.84 min | 0.1 |
| | | | | F; H | | method E | |
| 30 | F | CH₃ | NCH₃ | H; H; H; | 435 | 0.85 min | 1.0 |
| | | | | H; H | | method C | |
| 31 | F | CH₃ | NC₂H₅ | H; H; H; | 449 | 0.85 min | 0.2 |
| | | | | H; H | | method C | |
| 32 | F | CH₃ | NC₂H₅ | H; H; F; | 467 | 0.89 min | 0.2 |
| | | | | H; H | | method D | |
| 33 | H | CH₃ | O | CH₃; H; H; | 452 | 1.39 min | 2.2 |
| | | | | H; Cl | | method J | |
| 34 | H | CH₃ | O | CH₂CH₃; H; | 450 | 1.38 min | 0.3 |
| | | | | H; H; F | | method L | |
| 35 | H | CH₃ | O | CH₂CH₃; H; | 468 | 1.43 min | 0.3 |
| | | | | F; H; F | | method L | |
| 36 | H | CH₃ | O | H; H; H; | 438 | 1.32 min | 0.2 |
| | | | | Cl; H | | method M | |
| 37 | F | CH₃ | O | CH₃; H; H; | 470 | 1.46 min | 2.2 |
| | | | | H; Cl | | method J | |
| 38 | F | CH₃ | O | CH₂CH₃; H; H; | 468 | 0.88 min | 0.6 |
| | | | | H; F | | method E | |
| 39 | F | CH₃ | O | H; H; H; | 456 | 1.40 min | 0.4 |
| | | | | Cl; H | | method M | |
| 40 | F | CH₃ | NC₂H₅ | H; F; H; | 467 | 0.87 min | 0.4 |
| | | | | H; H | | method E | |
| 41 | F | CH₃ | NC₂H₅ | H; H; H; | 467 | 0.87 min | 0.1 |
| | | | | F; H | | method E | |
| 42 | F | CH₃ | N(CH₂)₂— | H; H; H; | 481 | 0.89 min | 0.1 |
| | | | CH₃ | F; H | | method E | |
| 43 | F | CH₃ | O | CH₂OCH₃; H; H; | 466 | 0.86 min | 2.3 |
| | | | | H; H | | method D | |
| 44 | F | CH₃ | O | CH₃; H; Cl; | 470 | 0.95 min | 0.3 |
| | | | | H; H | | method D | |
| 45 | F | CH₃ | O | CH₃; H; Br; | 514 | 0.96 min | 0.3 |
| | | | | H; H | | method D | |
| 46 | F | CH₃ | S | CH₃; H; F; | 470 | 0.88 min | 4.5 |
| | | | | H; H | | method D | |
| 47 | F | CH₃ | O | CH₂CH₃; H; F; | 468 | 0.94 min | 0.9 |
| | | | | H; H | | method D | |
| 48 | H | CH₃ | O | CH₂CH₃; H; F; | 450 | 0.85 min | 1.2 |
| | | | | H; H | | method E | |
| 49 | F | CH₃ | O | CH₃; H; OCH₃; | 466 | 1.30 min | 0.1 |
| | | | | H; H | | method F | |
| 50 | F | CH₃ | O | CH₃; H; H; | 454 | 1.32 min | 0.4 |
| | | | | H; F | | method F | |
| 51 | F | CH₃ | NCH₃ | H; H; F; | 453 | 1.28 min | 0.4 |
| | | | | H; H | | method F | |
| 52 | F | CH₃ | O | (CH₂)₂CH₃; H; F; | 482 | 0.87 min | 0.7 |
| | | | | H; H | | method D | |
| 53 | F | CH₃ | N(CH₂)₃— | H; H; H; | 477 | 1.41 min | 0.2 |
| | | | CH₃ | H; H | | method O | |
| 54 | F | CH₃ | N(CH₂)₂— | H; H; F; | 481 | 0.92 min | 0.2 |
| | | | CH₃ | H; H | | method D | |
| 55 | F | CH₃ | N(CH₂)₃— | H; H; F; | 495 | 0.91 min | 0.1 |
| | | | CH₃ | H; H | | method E | |
| 56 | F | CH₃ | N(CH₂)₂— | H; H; H; | 463 | 0.87 min | 0.2 |
| | | | CH₃ | H; H | | method E | |

TABLE 2
Compounds of formula Ib″

(Ib″)

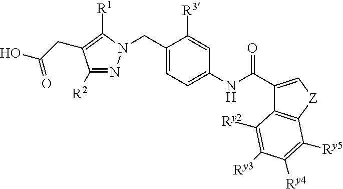

| | | | | R^{y1}; R^{y2}; | MS | Retention | Ki |
|---|---|---|---|---|---|---|---|
| Cmpd | R¹, R² | R^{3′} | Z | R^{y3}; R^{y4}; R^{y5} | [M + H]⁺ | Time | [nM] |
| 57 | CH₃ | H | O | H; H; H; H; H | 404 | 1.29 min | 8.8 |
| | | | | | | method | |
| | | | | | | B | |
| 58 | CH₃ | H | NH | H; H; H; H; H | 403 | 1.14 min | 37.2 |
| | | | | | | method | |
| | | | | | | B | |
| 59 | CH₃ | H | S | H; H; H; H; H | 420 | 1.32 min | 3.8 |
| | | | | | | method | |
| | | | | | | B | |

1. A method of treating or preventing hairloss in a patient in need thereof, the method comprising administering to the patient an effective amount of a compound of formula (Ia) or (Ib) or a pharmaceutically acceptable salt thereof 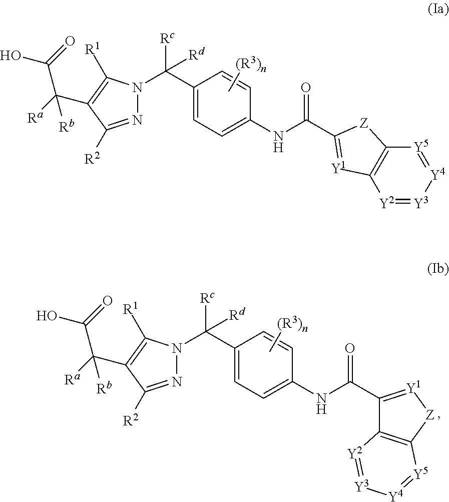
wherein:
R^{a} and R^{b} are each independently hydrogen, hydroxy, halogen, C₁-C₆-alkyl, C₁-C₆ haloalkyl, C₁-C₆-alkoxy, C₁-C₆-haloalkoxy, or C₃-C₈-cycloalkyl, or R^{a} and R^{b} together with the carbon atom they are bound to form a carbonyl group, or R^{a} and R^{b} together with the carbon atom they are bound to form a 3- to 8-membered ring, wherein the ring optionally contains 1 or 2 heteroatoms selected from O, N, and S as ring members and wherein the ring members of the ring are optionally independently substituted by hydroxy, halogen, C₁-C₆-alkyl, C₁-C₆-haloalkyl, C₁-C₆-alkoxy, C₁-C₆-haloalkoxy, or C₃-C₈-cycloalkyl;
R^{c} and R^{d} are each independently hydrogen, hydroxy, halogen, C₁-C₆-alkyl, C₁-C₆-haloalkyl, C₁-C₆-alkoxy, C₁-C₆-haloalkoxy, or C₃-C₈-cycloalkyl, or R^{c} and R^{d} together with the carbon atom they are bound to form may form a carbonyl group, or R^{c} and R^{d} together with the carbon atom they are bound to form a 3- to 8-membered ring, wherein the ring optionally contains 1 or 2 heteroatoms selected from O, N, and S as ring members and wherein the ring members of the ring are optionally independently substituted by hydroxy, halogen, C₁-C₆-alkyl, C₁-C₆-haloalkyl, C₁-C₆-alkoxy, C₁-C₆-haloalkoxy, or C₃-C₈-cycloalkyl;
Y¹, Y², Y³, Y⁴, and Y⁵ are each independently selected from N and CR^{y}, wherein each R^{y} is independently selected from H, hydroxy, halogen, cyano, nitro, SF₅, C(O)NR^{f}R^{g}, C₁-C₆-alkyl, hydroxy-C₁-C₆-alkyl, C₁-C₆-alkoxy-C₁-C₆-alkyl, C₃-C₈-cycloalkyl, C₁-C₆-haloalkyl, C₁-C₆-alkoxy, C₁-C₆-alkoxy-C₁-C₆-alkoxy, C₁-C₆-haloalkoxy, C₃-C₈-cycloalkoxy, C₁-C₆-alkylamino, di-C₁-C₆-alkylamino, C₁-C₆-alkylsulfonyl, phenyl, phenoxy, 5- or 6-membered heterocyclyl, and 5- or 6-membered heterocyclyloxy, wherein R^{f} and R^{g} are independently selected from H, C₁-C₆-alkyl, C₁-C₆-haloalkyl, C₃-C₈-cycloalkyl, C₃-C₈-cycloalkenyl, and 5- or 6-membered heterocyclyl or R^{f} and R^{g} together with the nitrogen atom to which they are bound form a cyclic amine optionally comprising a further heteroatom selected from O, N, and S as a ring member;
Z is O, S, or NR^{z}, wherein R^{z} is H, C₁-C₆-alkyl, or benzyl;
R¹ and R² are each independently selected from H, halogen, C₁-C₆-alkyl, C₂-C₆-alkenyl, C₂-C₆-alkynyl, C₁-C₆-alkoxy, C₁-C₆-alkylthio, —NR^{f}R^{g}, C₃-C₈-cycloalkyl, C₃-C₈-cycloalkyl-C₁-C₆-alkyl, C₃-C₈-cycloalkyl-C₂-C₆-alkenyl, C₃-C₈-cycloalkenyl, C₃-C₈-cycloalkenyl-C₁-C₆-alkyl, C₃-C₈-cycloalkenyl-C₂-C₆-alkenyl, phenyl, phenyl-C₁-C₆-alkyl, phenyl-C₂-C₆-alkenyl, naphthyl, naphthyl-C₁-C₆-alkyl, naphthyl-C₂-C₆-alkenyl, heterocyclyl, heterocyclyl-C₁-C₆-alkyl, and heterocyclyl-C₂-C₆-alkenyl,
wherein
the C₁-C₆-alkyl, C₂-C₆-alkenyl, and C₂-C₆-alkynyl moieties in R¹ and R² are unsubstituted or carry at least one substituent selected from hydroxy, halogen, cyano, nitro, C₁-C₆-alkoxy, C₁-C₆-haloalkoxy, C₁-C₆-alkylamino, di C₁-C₆-alkylamino, and C₁-C₆-alkylsulfonyl,
two radicals bound to the same carbon atom of the C₁-C₆-alkyl, C₂-C₆-alkenyl, and C₂-C₆-alkynyl moieties in R¹ and R² together with the carbon atom form a carbonyl group,
the C₃-C₈-cycloalkyl, cycloalkenyl, phenyl, naphthyl, and heterocyclyl moieties in R¹ and R² are unsubstituted or carry at least one substituent selected from hydroxy, halogen, cyano, nitro, C₁-C₆-alkyl, C₃-C₈-cycloalkyl, C₁-C₆-haloalkyl, C₁-C₆-alkoxy, C₁-C₆-haloalkoxy, C₁-C₆-alkylamino, di-C₁-C₆-alkylamino, C₁-C₆-alkylsulfonyl, phenyl, and 5- or 6-membered hetaryl,
two radicals bound to the same carbon atom of the C₃-C₈-cycloalkyl, C₃-C₈-cycloalkenyl, and heterocyclyl moieties of R¹ and R² together with the carbon atom form a carbonyl group, and
R^{f} and R^{g} are each independently H, C₁-C₆-alkyl, C₁-C₆-haloalkyl, C₃-C₈-cycloalkyl, C₃-C₈-cycloalkenyl, or heterocyclyl, or
R^{f} and R^{g} together with the nitrogen atom to which they are bound form a cyclic amine optionally comprising a further heteroatom selected from O, N, and S as a ring member;
n is 0, 1, 2, or 3; and
R³ are each independently halogen, C₁-C₆-alkyl, C₁-C₆-haloalkyl, C₁-C₆-alkoxy, C₁-C₆-haloalkoxy, or C₃-C₈-cycloalkyl.

2. The method according to claim 1, wherein R^{a} and R^{b} are both hydrogen.

3. The method according to claim 1, wherein R^{c} and R^{d} are both hydrogen.

4. The method according to claim 1, wherein Y¹ is CR^{y1}, wherein R^{y1} is H, C₁-C₆-alkyl, C₁-C₆-alkoxy-C₁-C₆-alkyl, or C₁-C₆-haloalkyl.

5. The method according to claim 1, wherein Y² is CR^{y2}, Y³ is CR^{y3}, Y⁴ is CR^{y4}, and Y⁵ is CR^{y5}, wherein R^{y2}, R^{y3}, R^{y4}, and R^{y5} are each independently H, halogen, C₁-C₆-alkoxy, C₁-C₆-alkoxy-C₁-C₆-alkoxy, or C₁-C₆-haloalkoxy.

6. The method according to claim 1, wherein Z is O.

7. The method according to claim 1, wherein Z is S.

8. The method according to claim 1, wherein Z is NR^{z}.

9. The method according to claim 1, wherein R¹ and R² are each C₁-C₄-alkyl.

10. The method according to claim 1, wherein n is 0 or 1.

11. The method according to claim 1, wherein R³ are independently halogen.

12. The method according to claim 1, wherein the compound of formula (I) or a pharmaceutically acceptable salt thereof is administered in the form of a pharmaceutical composition a compound of formula (Ia) or (Ib) or a pharmaceutically acceptable salt thereof and a pharmaceutical excipient.

13. The method according to claim 12, wherein the pharmaceutical composition is a tablet, capsule, pill, solution, suspension, dispersion, emulsion, or suppository.

14. The method according to claim 12, wherein the pharmaceutical composition is a solution, suspension, emulsion, dispersion, cream, ointment, gel, lotion, shampoo, or aerosol.

15. The method according to claim 1, wherein the hairloss is related to androgenic alopecia.

16. The method according to claim 14, wherein the hairloss is related to androgenic alopecia.

17. The method according to claim 15, wherein the androgenic alopecia is male pattern baldness or female pattern baldness.

18.-21. (canceled)
